(19) United States
(12) Patent Application Publication
(10) Pub. No.: US 2017/0326193 A1
(43) Pub. Date: Nov. 16, 2017

(54) BUCHU PREPARATIONS

(71) Applicant: CAPE KINGDOM NUTRACEUTICALS (PTY) LTD, Big Bay (ZA)

(72) Inventor: Michael Duncombe STANDER, Big Bay (ZA)

(21) Appl. No.: 15/532,380

(22) PCT Filed: Dec. 1, 2015
(86) PCT No.: PCT/IB2015/059238

§ 371 Date: Jun. 1, 2017

(30) Foreign Application Priority Data
Dec. 1, 2014 (ZA) 2014/08792

(51) Int. Cl. A61K 36/75; A61K 9/00; A61K 9/06; A61K 47/14

(52) U.S. Cl. CPC A61K 36/75; A61K 47/14; A61K 9/0014; A61K 9/06

(57) ABSTRACT

The invention discloses a topical composition, which includes at least one buchu oil extract and at least one buchu water or aqueous extract. The composition includes at least one active ingredient originating from at least one buchu oil extract and at least one buchu water or aqueous extract or bio-active fraction thereof in a pharmaceutically acceptable form. The Buchu oil extract includes about 60% of a betulina buchu variety and about 40% of a crenulata buchu variety. The Buchu oil extract forms about 0.25 weight % of the entire topical formulation. The invention also extends to a sports recovery drink composition, which includes buchu oil, glutamine and beta-carotene.

### FIELD OF INVENTION

The present invention relates to buchu preparations.

More particularly, the present invention relates to buchu preparations including a topical formulation including Buchu plant material extracts, a sports recovery drink composition including buchu oil, and a buchu water composition including a buchu plant oil extract and spring water or mineral water.

### BACKGROUND TO INVENTION

Buchu is one of the best known medicinal plants of South Africa and is indigenous to the Cedarberg Mountains and surrounding areas. Despite its popularity little scientific evidence exists about the various medicinal uses of this small fynbos shrub from the family Rustaceae. The two primary species of Buchu used commercially are Agasthoma betulina (round-leaf Buchu) and Agathosma crenulata (oval-leaf Buchu). Besides its medicinal properties, Buchu oil is also used in the flavourant and fragrance industry, currently the largest commercial use thereof.

Buchu oil is typically prepared in a (high vacuum) low steam distillation process in which the Buchu oil required for the commercial market is extracted from the plant material and separated from the by-products of this steam distillation process.

It is an object of the invention to suggest buchu preparations of Buchu plant material extracts.

### SUMMARY OF INVENTION

According to the invention, there is provided a topical formulation including at least one buchu oil extract and at least one buchu water or aqueous extract.

Also according to the invention, there is provided a topical formulation including at least one active ingredient originating from at least one buchu oil extract and at least one buchu water or aqueous extract or bio-active fraction thereof in a pharmaceutically acceptable form.

The invention also extends to a therapeutic topical composition, pharmaceutical topical composition or modified buchu oil extract and buchu water or aqueous extract or bio-active fraction thereof including, for use in a method of inducing an anti-bacterial or anti-viral response in a mammal, preferably a human, in need thereof.

The invention extends further to the use of at least one buchu oil extract and at least one buchu water or aqueous extract in the manufacture of a medicament.

The invention extends further to the use of at least one buchu oil extract and at least one buchu water or aqueous extract in the manufacture of a medicament for use in a method of inducing an anti-bacterial or anti-viral response in a mammal, preferably a human, in need thereof.

According to a further aspect of the invention, there is provided a method of treating a bacterial or viral infection comprising administering to a patient in need thereof a topical formulation including at least one buchu oil extract and at least one buchu water or aqueous extract.

The topical formulation may be a therapeutic formulation.

The topical formulation may be a gel or cream.

The topical formulation may include carbopal ultrez-10.

The topical formulation may include Liponic EG-1.

The topical formulation may include Liposorb 1-20.

The topical formulation may include tea.

The topical formulation may include Microcare DMP.

The topical formulation may substantially include the following weight composition:

| | | |
|---|---|---|
| (a) | Buchu oil extract | 0.25% |
| (b) | Buchu water or aqueous extract | 91.75 |
| (c) | Carbopal Ultrez-10 | 1% |
| (d) | Liponic EG-1 | 2.5% |
| (e) | Liposorb I20 | 2.5% |
| (f) | Tea 99% | 1% |
| (g) | Microcare DMP | 1% |

The Buchu oil extract may include about 60% of a betulina buchu variety and about 40% of a crenulata buchu variety.

The Buchu oil extract may form about 0.25 weight % of the entire topical formulation.

The Buchu water or aqueous extract may form about 91.75% of the total topical formulation.

The Buchu water or aqueous extract may be obtained from a steam vacuum distillation process used for extracting Buchu oil from Buchu plant material and during which an aqueous portion was separated from the oil portion to provide a fraction of soluble oil of buchu.

The Buchu water or aqueous extract may include plant material of a betulina buchu variety and of a crenulata buchu variety.

The topical formulation may be used to treat at least one of the following conditions:
(a) Reduction of chronic pain;
(b) Reduction of inflammation (acute and chronic);
(c) Treatment of arthritis, rheumatism and other joint destroying conditions;
(d) Prevention of swelling and bruising following trauma of soft tissues;
(e) Treatment of infections (both gram negative and gram position, including microbial testing);
(f) Treatment of burns;
(g) Treatment of eczema;
(h) Post skin surgery; and
(i) Vaginal spermicide for the prevention of HIV

The topical formulation may be used to treat at least one of the following bacteria:
(a) E coli Nissle 1917;
(b) Enterococcus faecalis;
(c) Pseudomonas aeruginosa;
(d) Proteus mirabilis;
(e) Candida albicans;
(f) Staph aureus;
(g) MRSA;
(h) Streptococcus pyogenes;
(i) Aspergillus spp;
(j) Bacteriodes spp;
(k) Coliforms;
(l) B subtilis;
(m) A marcescens; and
(n) S pombe.

The Buchu oil extract may be obtained from the species Agasthoma betulina (round-leaf Buchu) and/or Agathosma crenulata (oval-leaf Buchu).

The Buchu water or aqueous extract may be obtained from the species Agasthoma betulina (round-leaf Buchu) and/or Agathosma crenulata (oval-leaf Buchu).

According to the invention, there is provided a sports recovery drink composition including buchu oil, glutamine and beta-carotene.

The buchu oil may include at least one buchu oil extract in combination with spring water and/or mineral water.

The composition may be a herbal composition.

The composition may have health benefits.

The composition may have rehydration benefits.

The composition may have anti-inflammatory and/or metabolic benefits.

The Buchu oil extract may form about 0.01-10 weight % of the composition.

The Buchu oil extract may be obtained from a steam vacuum distillation process in which extraction from Buchu plant material occurs.

The Buchu oil extract may be derived from plant material of a betulina buchu variety and/or of a crenulata buchu variety.

The Buchu oil extract may be obtained from the species Agasthoma betulina (round-leaf Buchu) and/or Agathosma crenulata (oval-leaf Buchu).

According to the invention, there is provided a buchu water composition including at least one buchu oil extract in combination with spring water and/or mineral water.

The buchu water composition may be a herbal composition.

The buchu water composition may have health benefits.

The Buchu oil extract may form about 0.01-10 weight % of the buchu water composition.

The Buchu oil extract may be obtained from a steam vacuum distillation process in which extraction from Buchu plant material occurs.

The Buchu oil extract may be derived from plant material of a betulina buchu variety and/or of a crenulata buchu variety.

The Buchu oil extract may be obtained from the species Agasthoma betulina (round-leaf Buchu) and/or Agathosma crenulata (oval-leaf Buchu).

### BRIEF DESCRIPTION OF EXAMPLE

The invention will now be described by way of example.

In accordance with the invention, there is provided a topical formulation including at least one buchu oil extract and at least one buchu water or aqueous extract.

The topical formulation includes at least one active ingredient originating from the buchu oil extract or the buchu water or aqueous extract or bio-active fraction thereof in a pharmaceutically acceptable form.

The topical formulation is a therapeutic formulation in a gel or cream state.

The topical formulation substantially includes the following weight composition:

| | | |
|---|---|---|
| (a) | Buchu oil extract | 0.25% |
| (b) | Buchu water or aqueous extract | 91.75 |
| (c) | Carbopal Ultrez-10 | 1% |
| (d) | Liponic EG-1 | 2.5% |
| (e) | Liposorb I20 | 2.5% |
| (f) | Tea 99% | 1% |
| (g) | Microcare DMP | 1% |

The Buchu oil extract includes about 60% of a betulina buchu variety and about 40% of a crenulata buchu variety.

The Buchu water or aqueous extract is obtained from a high vacuum low steam distillation process traditionally used for extracting Buchu oil from Buchu plant material to obtain the Buchu oil extract. The aqueous portion is separated from the oil portion, and treated in order to remove impurities and other non-water-soluble components, including Buchu oil residue. This modified aqueous Buchu extract is hereinafter referred to as the “Buchu water or aqueous extract” for convenience.

The Buchu water or aqueous extract includes plant material of a betulina buchu variety and of a crenulata buchu variety.

The topical formulation is adapted to be used to treat at least one of the following conditions:
(a) Reduction of chronic pain;
(b) Reduction of inflammation (acute and chronic);
(c) Treatment of arthritis, rheumatism and other joint destroying conditions;
(d) Prevention of swelling and bruising following trauma of soft tissues;
(e) Treatment of infections (both gram negative and gram position, including microbial testing);
(f) Treatment of burns;
(g) Treatment of eczema;
(h) Post skin surgery; and
(i) Vaginal spermicide for the prevention of HIV

The topical formulation is adapted to be used to treat at least one of the following bacteria:
(a) E coli Nissle 1917;
(b) Enterococcus faecalis;
(c) Pseudomonas aeruginosa;
(d) Proteus mirabilis;
(e) Candida albicans;
(f) Staph aureus;
(g) MRSA;
(h) Streptococcus pyogenes;
(i) Aspergillus spp;
(j) Bacteriodes spp;
(k) Coliforms;
(l) B subtilis;
(m) A marcescens; and
(n) S pombe.

The Buchu oil extract is obtained from the species Agasthoma betulina (round-leaf Buchu) and/or Agathosma crenulata (oval-leaf Buchu).

The Buchu water or aqueous extract is obtained from the species Agasthoma betulina (round-leaf Buchu) and/or Agathosma crenulata (oval-leaf Buchu).

The invention thus also extends to a therapeutic topical composition, pharmaceutical topical composition or modified buchu oil extract and the buchu water or aqueous extract or bio-active fraction thereof including, for use in a method of inducing an anti-bacterial or anti-viral response in a mammal, preferably a human, in need thereof.

The invention extends further to the use of at least one buchu oil extract and at least one buchu water or aqueous extract in the manufacture of a medicament.

The invention extends further to the use of at least one buchu oil extract and at least one buchu water or aqueous extract in the manufacture of a medicament for use in a method of inducing an anti-bacterial or anti-viral response in a mammal, preferably a human, in need thereof.

According to a further aspect of the invention, there is provided a method of treating a bacterial or viral infection comprising administering to a patient in need thereof a topical formulation including at least one buchu oil extract and at least one buchu water or aqueous extract.

In accordance with the invention, there is provided a sports recovery drink composition including buchu oil, glutamine and beta-carotene.

The buchu oil include at least one buchu oil extract in combination with spring water or mineral water.

The composition is a herbal composition and has health benefits.

The composition has rehydration benefits.

The composition has anti-inflammatory and/or metabolic benefits.

The Buchu oil extract forms about 0.01-10 weight % of the composition.

The Buchu oil extract is obtained from a steam vacuum distillation process in which extraction from Buchu plant material occurs.

The Buchu oil extract is derived from plant material of a betulina buchu variety and of a crenulata buchu variety.

The Buchu oil extract is obtained from the species Agasthoma betulina (round-leaf Buchu) and/or Agathosma crenulata (oval-leaf Buchu).

In accordance with the invention, there is provided a buchu water composition including at least one buchu oil extract in combination with spring water or mineral water.

The buchu water composition is a herbal composition and has health benefits.

The Buchu oil extract forms about 0.01-10 weight % of the buchu water composition.

The Buchu oil extract is obtained from a steam vacuum distillation process in which extraction from Buchu plant material occurs.

The Buchu oil extract is derived from plant material of a betulina buchu variety and of a crenulata buchu variety.

The Buchu oil extract is obtained from the species Agasthoma betulina (round-leaf Buchu) and/or Agathosma crenulata (oval-leaf Buchu).

1. A topical composition, which includes at least one buchu oil extract and at least one buchu water or aqueous extract.

2. The composition as claimed in claim 1, which includes at least one active ingredient originating from at least one buchu oil extract and at least one buchu water or aqueous extract or bio-active fraction thereof in a pharmaceutically acceptable form.

3. The composition as claimed in claim 1, which is a therapeutic formulation.

4. The composition as claimed in claim 1, which is a gel or cream.

5. The composition as claimed in claim 1, which includes carbopal ultrez-10, Liponic EG-1 , Liposorb 1-20, tea and/or Microcare DMP.

6. The composition as claimed in claim 1, which includes the following components:
(a) Buchu oil extract
(b) Buchu water or aqueous extract
(c) Carbopal Ultrez-10
(d) Liponic EG-1
(e) Liposorb 120
(f) Tea 99%
(g) Microcare DMP

7. The composition as claimed in claim 6, which includes the following compositions:
(a) Buchu oil extract 0.25%
(b) Buchu water or aqueous extract 91 .75
(c) Carbopal Ultrez-10 1%
(d) Liponic EG-1 2.5%
(e) Liposorb 120 2.5%
(f) Tea 99% 1%
(g) Microcare DMP 1%

8. The composition as claimed in claim 1, in which the Buchu oil extract includes about 60% of a betulina buchu variety and about 40% of a crenulata buchu variety.

9. The composition as claimed in claim 1, in which the Buchu oil extract forms about 0.25 weight % of the entire topical formulation.

10. The composition as claimed in claim 1, in which the Buchu water or aqueous extract forms about 91 .75% of the total topical formulation.

11. (canceled)

12. The composition as claimed in claim 1, in which the Buchu water or aqueous extract includes plant material of a betulina buchu variety and of a crenulata buchu variety.

13-16. (canceled)

17. A sports recovery drink composition, which includes buchu oil, glutamine and beta-carotene.

18. The composition as claimed in claim 17, in which the buchu oil includes at least one buchu oil extract in combination with spring water and/or mineral water.

19. The composition as claimed in claim 17, is a herbal composition.

20. (canceled)

21. The composition as claimed in claim 17, in which the Buchu oil extract forms about 0.01 -10 weight % of the composition.

22. A method of manufacturing the composition as claimed in claim 17, comprising extracting in which the Buchu oil extract is from Buchu plant via a steam vacuum distillation process.

23. The method of claim 22, wherein the Buchu oil extract is derived from plant material of a betulina buchu variety and/or of a crenulata buchu variety.

24. The method of claim 22, owherein the Buchu oil extract is obtained from the species Agasthoma betulina (round-leaf Buchu) and/or Agathosma crenulata (oval-leaf Buchu).

25. A buchu water composition, which includes at least one buchu oil extract in combination with spring water and/or mineral water.

26. The composition as claimed in claim 25, which is a herbal composition.

27. (canceled)

28. The composition as claimed in claim 25, in which the Buchu oil extract forms about 0.01 -10 weight % of the buchu water composition.

29. A method for manufacturing the composition of claim 25, comprising extracting the Buchu oil extract from Buchu plant via a steam vacuum distillation process in.

30. The method of claim 25, in which the Buchu oil extract is derived from plant material of a betulina buchu variety and/or of a crenulata buchu variety.

31. The method of claim 25, in which the Buchu oil extract is obtained from the species Agasthoma betulina (round-leaf Buchu) and/or Agathosma crenulata (oval-leaf Buchu).

32. A therapeutic topical composition, pharmaceutical topical composition or modified buchu oil extract and buchu water or aqueous extract or bio-active fraction thereof for use in a method of inducing an anti-bacterial or anti-viral response in a mammal, preferably a human, in need thereof.

33-34. (canceled)

35. A method of treating a bacterial or viral infection, which includes the step of administering to a patient in need thereof a topical formulation including at least one buchu oil extract and at least one buchu water or aqueous extract.

36-43. (canceled)
